(19) United States
(12) Patent Application Publication
(10) Pub. No.: US 2003/0221977 A1
(43) Pub. Date: Dec. 4, 2003

(54) Dental implant vial with activation mechanism

(76) Inventors: Ajay Kumar, Palmdale, CA (US); Ines Aravena, Camarillo, CA (US); Jeff Bassett, Vista, CA (US); Mathew Saccomanno III, Oceanside, CA (US)

Correspondence Address: Attention: Kenneth S. Barrow, Centerpulse USA Inc., 12 Greenway Plaza, Suite 1000, Houston, TX, 77046, US

(21) Appl. No.: 10/372,365

(22) Filed: Feb. 21, 2003

Related U.S. Application Data
(60) Provisional application No. 60/385,801, filed on Jun. 4, 2002.

(51) Int. Cl. A61B 19/02

(52) U.S. Cl. 206/63.5

(57) ABSTRACT

A vial for housing a dental implant and having an active retention mechanism that is moveable to one of two positions. In a first position, the retention mechanism firmly holds the implant during transportation or other periods of non-use. In a second position, the retention mechanism releases the implant so it can be removed from the vial.

### FIELD OF THE INVENTION

The disclosure herein relates generally to dental implants in the field of odontology and, more particularly, to a new vial with an activation mechanism for holding and releasing a dental implant.

### BACKGROUND OF THE INVENTION

Dental implants are typically packaged and shipped in a vial and surrounded with an outer package. The vial maintains the implant in a sterile environment and is opened just before the implant is needed during a dental implantation surgical procedure.

The vial has a generally elongated, cylindrical configuration with a closed bottom. end and an open top end. A cap connects to the top end and can be opened or removed to give access to an internal cavity in the vial. The cap and vial provide a protective, sealed enclosure for the dental implant.

Typically, the implant is suspended or held in the internal cavity of the vial. Prior designs have various ways to hold the implant in the internal cavity. The coronal end of the implant, for example, can be seated against an internal shoulder in the vial. Alternatively, the implant can be frictionally held in the cavity with an o-ring or other frictional engagement mechanism. Other designs provide a recess in the cap, and the end of the implant is press-fit or frictionally engaged in this recess.

Implants are removed from the vial with various techniques, depending on the particular design of the vial and holding mechanism. In many of these techniques, friction is the key force. In order to remove an implant frictionally suspended in a vial, the cap is opened to provide access to the internal cavity and implant. A driving tool frictionally engages the coronal end of the implant and pulls it upwardly, away from the vial. When the upward removal force is greater than the frictional retentive force, the implant dislodges and separates from the vial. The implant is then transported to the osteotomy and driven into the jawbone with the driving tool. When installation is complete, the driving tool is disengaged from the implant. Here again, frictional forces play a key role. When the upward removal force of the driving tool is greater than the frictional retentive force, the driving tool dislodges and separates from the implant, leaving the implant embedded in the jawbone.

Unfortunately, the designs of many dental implant vials have significant disadvantages. As one example, a relatively large frictional force is used to hold the implant in the vial; and this same frictional force must be overcome to remove the implant from the vial. Obviously, the retentive or engagement force between the driving tool and the implant must be greater than the frictional force holding the implant in the vial. This retentive force, then, can be relatively large. Further, this same retentive force must be overcome to disengage the driving tool from the implant while it is situated in the osteotomy.

A large removal force between the driving tool and implant should not be applied to an implant once it is installed and positioned in the osteotomy. Some parts of the jawbone, especially the posterior maxilla, are formed of softer bone. When the implant is correctly positioned in this bone, external forces can readily disrupt the position of the implant or deleteriously affect the commencement of osseointegration.

As another disadvantage, implants are subject to vibrations and other forces while in the vial during shipping; and these forces can cause the implant to move or rattle, creating an unwanted noise. Some implants have very precise coatings applied to the external surface, and rattling or bumping against the internal cavity of the vial can affect the coating.

As a further disadvantage, some implants cannot readily be replaced back into a vial once removed. If an implant is suspended in the vial with a retention member, such as an o-ring or clip, the retention member can become lost or damaged once the implant is removed from the vial. Further yet, after the retention member is removed, it may not easily go back into place.

It therefore would be advantageous to have a new dental implant vial that overcame disadvantages associated with prior designs and offered advantages over these prior designs.

### SUMMARY OF THE INVENTION

The present invention is directed toward an improved vial for housing a dental implant. The vial has an active retention mechanism that firmly holds the implant during transportation or other periods of non-use. This retention mechanism can be activated to release the implant when it is ready to be removed from the vial.

One important advantage of the present invention is that the active retention mechanism holds and retains the implant in the vial. The retention mechanism can be moved or actuated to one of two positions. In a first or closed position, the retention mechanism firmly grips and holds the implant in the vial. The implant is not free to move in the vial while in this position. In a second or open position, the retention mechanism completely disengages from the implant. Now, the implant can be readily removed from the vial with no frictional or retentive forces holding the implant in or to the vial.

The vial of the present invention has many advantages. As noted, the implant is firmly held or retained in the vial when not being used. When the implant is ready to be used, the retention mechanism is activated and the implant is released. No frictional or retentive forces now hold the implant in the vial, so the implant can be removed with a relatively low or small frictional force. Specifically, a driving tool can be frictionally engaged on the coronal end of the implant to lift and pull it from the vial. This frictional retentive force between the driving tool and implant can be relatively small since the implant is not restrained in the vial with retaining forces. In fact, this retentive force can be low enough to hold the weight of the implant and ensure it cannot easily fall during transportation to and insertion in the osteotomy.

With the present invention, a large removal force between the driving tool and implant is not applied to the implant once it is installed and positioned in the osteotomy. Some parts of the jawbone, especially the posterior maxilla, are formed of softer bone. When the implant is correctly positioned in the bone, for instances the posterior maxilla, a small or light removal force is required to disengage the implant from the driving tool. This removal force will not disrupt the position of the implant or otherwise deleteriously affect the commencement of osseointegration.

As another advantage, the implant is firmly held in the vial when the retention mechanism is in a closed position. In this position, the implant will not rattle or repeatedly bump against an internal wall in the vial. Coatings on the surface of the implant are, thus, more likely to maintain integrity during shipping and transportation.

As yet another advantage, implants utilizing the present invention can readily be replaced back into a vial even after they are removed. Once the implant is removed, the retention mechanism remains in an open position. While the implant is being positioned back into the vial, the retention mechanism can be re-activated to the closed position and hold and retain the implant in a firm position.

The invention, accordingly, comprises the apparatus and method possessing the construction, combination of elements, and arrangement of parts that are exemplified in the following detailed description. For a fuller understanding of the nature and objects of the invention, reference should be made to the following detailed description taken in connection with the accompanying drawings.

### BRIEF DESCRIPTION OF THE DRAWINGS

FIG. 1 is an exploded view of a vial housing a retention mechanism according to the present invention.

FIG. 2 is a cross-sectional view of an assembled vial of FIG. 1.

FIG. 3 is a partial perspective view of the top of the housing of the vial.

FIG. 4 is a perspective view of an indexer and contra-indexer that form part of the vial.

FIG. 5 is a perspective view of a cradle that forms part of the vial.

FIG. 5A is an enlarged portion shown at arrow “A” of FIG. 5.

FIG. 6 is a perspective view of the cradle connected to the contra-indexer and indexer.

FIG. 6A is an enlarged portion shown at arrow “A” of FIG. 6.

FIG. 7 is a perspective view of a gripper that forms part of the vial.

FIG. 8 is a partial perspective view of the gripper connecting to the top of the housing of the vial.

FIG. 9 is a perspective view of the cradle, gripper, and housing connected together.

FIG. 10 is a cross-sectional view of the vial in a closed-position gripping a dental implant.

FIG. 11 is a cross-sectional view of the vial in an open-position with the dental implant being released.

FIG. 12 is a perspective view of a dental driving tool engaging with a coronal end of a dental implant in the vial.

FIG. 12A is an enlarged partial cross-sectional view shown at arrow “A” of FIG. 12.

### DETAILED DESCRIPTION

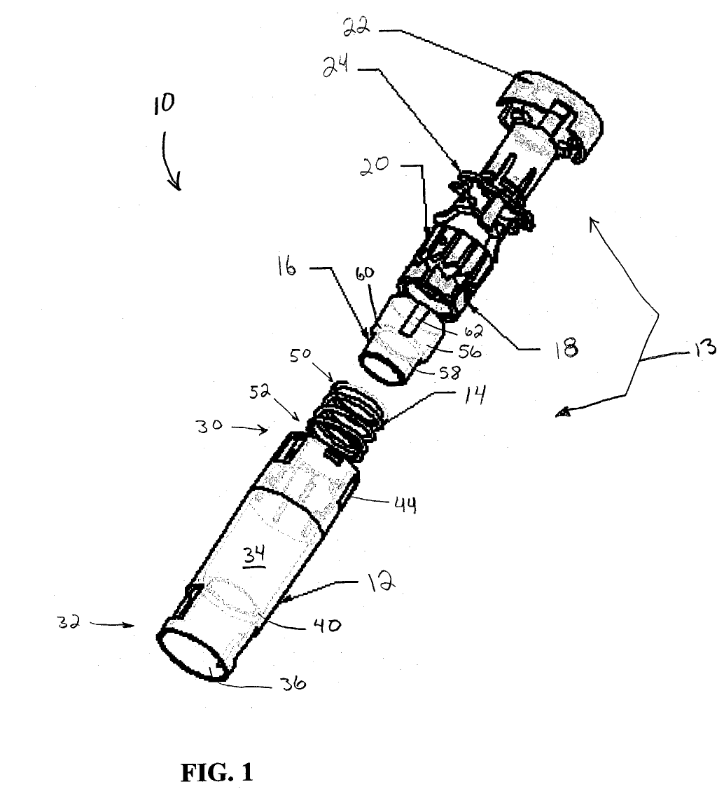

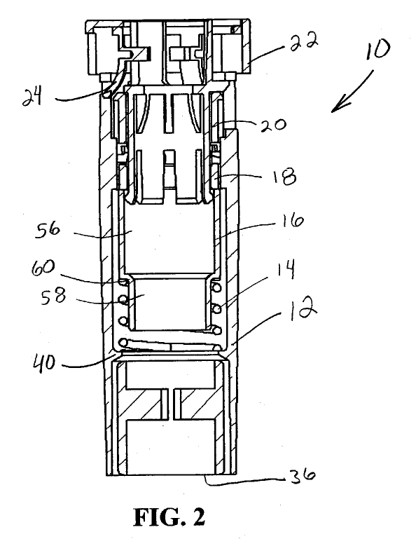

Looking to FIGS. 1 and 2, the vial 10 of the present invention is shown. The vial 10 has a housing 12 with a generally elongated cylindrical configuration that is adapted to house a dental implant. An active retention mechanism 13 fits in the housing and comprises several different components. These components include a biasing member 14, a piston 16, an indexer 18, a contra-indexer 20, a cradle 22, and a gripper 24.

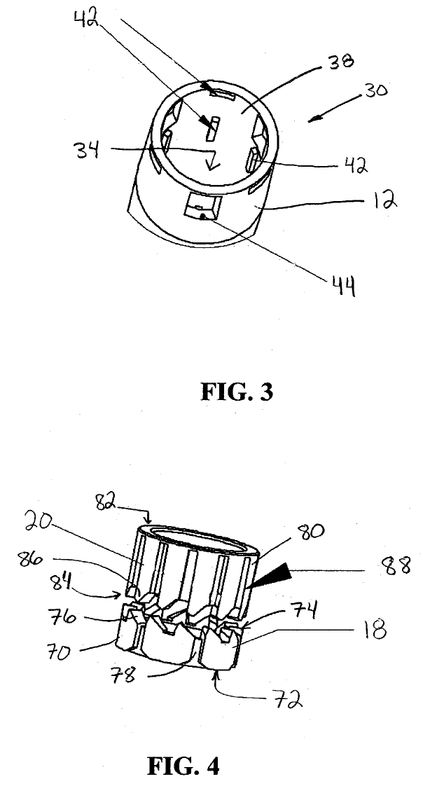

Looking also to FIG. 3, the housing 12 has a cylindrical shape with a body that extends from a proximal portion 30 to a distal portion 32. A hollow, cylindrical cavity 34 extends along the body and has a closed, imperforate end 36 at the distal end and an opening 38 at the proximal end. An internal shoulder 40 extends around the cavity 34. As best shown in FIG. 3, a plurality of guide rails or ribs 42 with an elongated rectangular shape extend along the cavity 34. Further, several rectangular or square shaped slots 44 extend through the body at the proximal portion 30.

Biasing member 14 is positioned inside cavity 34 and extends from a first end 50 to a second end 52, wherein the second end is adapted to abut against shoulder 40 of housing 14. The biasing member is shown as a helical or coiled spring, but it may have various configurations known to those skilled in the art.

The piston 16 generally is formed as a hollow cylinder with a top cylindrical portion 56 and a smaller, bottom cylindrical portion 58. Top and bottom portions form an external shoulder 60 that extends around the outer diameter of the body. Bottom portion 58 is adapted to fit inside the biasing member 14 so the first end 50 of biasing member 14 abuts against the shoulder 60 of piston 16. The biasing member is adapted to upwardly bias the piston when compressed. Several slots 62 are formed in the body of the piston.

Looking also to FIG. 4, the indexer 18 is shown in more detail. Indexer 18 has a body 70 with a hollow cylindrical configuration. A first end 72 has a flat surface, and a second end 74 has a plurality of teeth or gears 76. Longitudinal channels or slots 78 extend along the outer surface of body 70. These channels are adapted to receive and slideably engage the guide rails 42 of housing 14.

Contra-indexer 20 has a body 80 with a hollow cylindrical configuration. A first end 82 has a flat surface, and a second end 84 has a plurality of teeth or gears 86. These teeth are configured and adapted to mate and lock with corresponding teeth 76 on the indexer 18. Longitudinal channels or slots 88 extend along the outer surface of body 80. These channels are adapted to receive and slideably engage the guide rails 42 of housing 14.

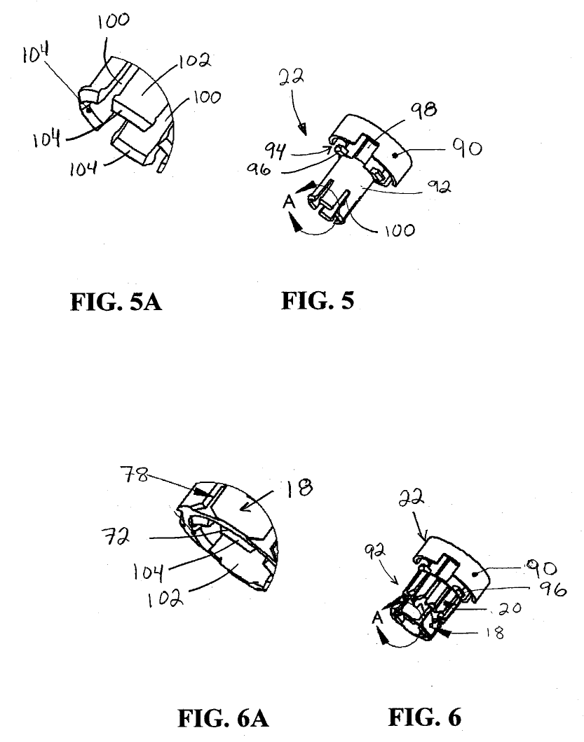

Looking now to FIGS. 5 and 5A, the cradle 22 is shown in more detail to have a cylindrical body with a top cylindrical portion 90 and a smaller, longer bottom cylindrical portion 92. Top portion 90 is formed as a cap or head with a cylindrical channel 94 circumferentially extending between the top and bottom portions. Several projections or tabs 96 extend outwardly from the outer surface of the bottom portion and are positioned in or adjacent the channel 94. Slots or recesses 98 are formed in the cap or top portion 90. A plurality of channels 100 are formed at the end of the bottom portion 92 and create a plurality of flexible fingers or arms 102. A lip or tap 104 is positioned at the end of each finger 102.

As shown in FIGS. 6 and 6A, the indexer 18 and contra-indexer 20 are adapted to be positioned around the bottom portion 92 of cradle 22. These components are captured between the tabs 96 and lip 104. As best shown in FIG. 6A, lip 104 fits under the end surface 72 of indexer 18. Flexible arms 102 may be flexed inwardly in a radial direction to receive the indexer and contra-indexer.

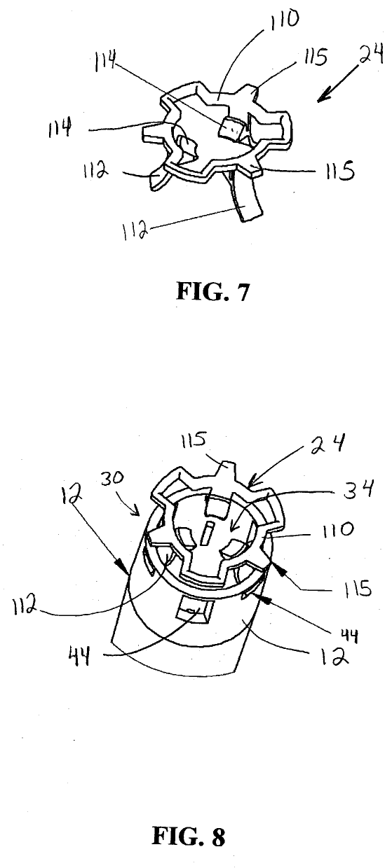

Turning now to FIG. 7, gripper 24 has a generally annular body 110 with a plurality of resilient curved legs or fingers 112 extending downwardly from the body. Each leg 112 has a foot or pad 114 that extends from a mid-section and inwardly toward a center of the body. A plurality of evenly spaced projections or tabs 115 extend outwardly from the body 110.

Looking to FIG. 8, gripper 24 is adapted to fit into the proximal portion 30 of housing 12. Tabs 115 of annular body 110 rest on top of an end surface of the housing 12, and legs 112 extend down into cavity 34 and project out and through slots 44.

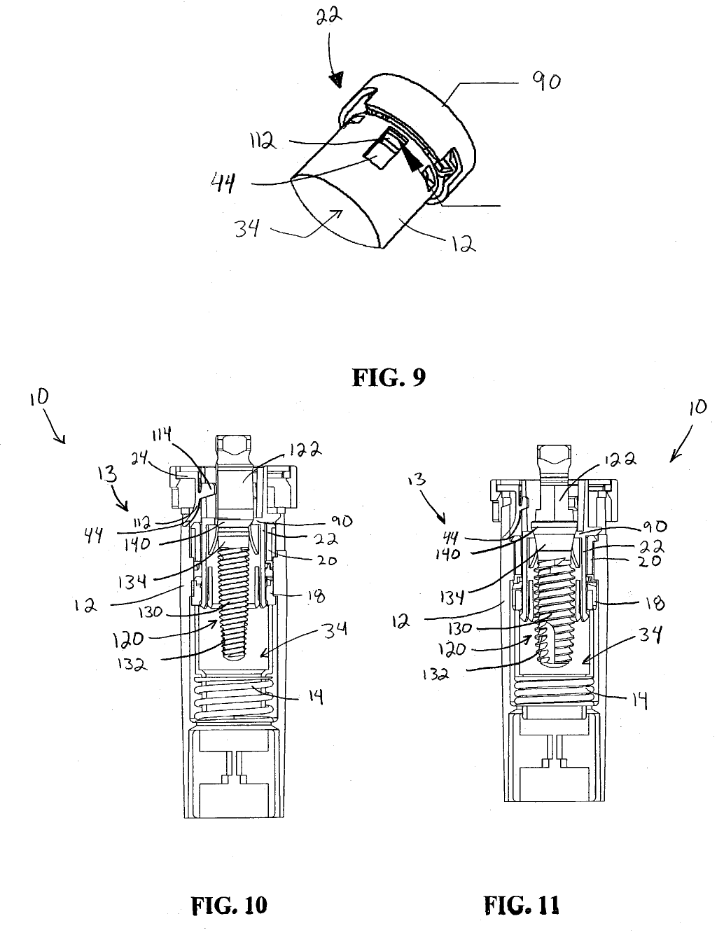

As shown in FIG. 9, the cradle 22 is adapted to fit over the end of housing 12. Specifically, the bottom portion 92 (FIG. 5) fits into cavity 34 until cap 90 covers opening 38 (FIG. 3). Legs 112 are positioned so they can flexibly extend outwardly through slots 44 of housing 12.

FIGS. 10 and 11 show the vial 10 assembled with a dental implant 120 and fixture mount transfer 122 positioned in cavity 34. The implant comprises a body or root portion 130 adapted to engage an osteotomy or alveolar cavity in the jawbone of a patient. The body includes external threads 132 adapted to engage bone in the osteotomy. The implant also includes a hexagonal recess or bore (not shown) at a top or coronal end 134. As is known in the art, this recess includes an internally threaded portion or socket that extends into the body portion of the implant.

The fixture mount 122 has a cylindrical body with a distal end 140 that abuts against the coronal end 134 of implant 120. A cavity extends through the body of the fixture mount and is adapted to receive a retaining. screw (not shown) that threadably connects with the implant 120.

The implant fits into the housing 12 of the vial 10 until the coronal end 134 abuts against the top surface of the head 90 of cradle 22. In this regard, the implant is suspended in the vial.

Various anti-rotational connections can be used to engage the implant and fixture mount. For example, this anti-rotational engagement could be provided as a hexagonal post or protrusion to engage a corresponding hexagonal recess. Further, as known to those skilled in the art, other anti-rotational connections (such as octagons, triangles, stars, splines, and the like) are also available.

As best shown in FIGS. 10 and 11, the retention mechanism 13 holds and retains the implant 120 in the vial and can be moved or actuated to one of two positions: A closed position and an open position. FIG. 10 illustrates the closed position in which the retention mechanism 13 firmly grips and holds the implant 120 in the housing 12. Specifically, the legs 112 of the gripper 24 project outwardly through slots 44 and abut against the outer surface of the fixture mount (or implant if no fixture mount is used). Pads or feet 114 of the gripper engage against the fixture mount (or implant). In the closed position, the biasing member 14 is not compressed, and the implant can be shipped and transported in the vial 10.

FIG. 11 illustrates the open position in which the retention mechanism has completely disengaged from the implant 120. Now, the implant can be readily removed from the vial with no frictional or retentive forces holding the implant in or to the vial. Specifically, the legs 112 of the gripper 24 are retracted through slots 44 and are disengaged from the fixture mount (or implant). In the open position, the biasing member 14 is compressed. Here, the indexer 18 and contra-indexer 20 abut each other so teeth 76 engage and lock with teeth 86 (FIG. 4). These teeth are interlocked to maintain the biasing member 14 in a compressed state and the vial 10 in the open position.

Another advantage of the present invention is that the implant can be removed from the vial and then replaced back into it. The retention mechanism can thus move back and forth between the open and closed positions. After the implant is removed, it can be positioned back into the housing and cradle. As the implant is pushed downwardly, the indexer 18 and contra-indexer 20 unlock and the biasing member 14 moves to an unbiased position. Simultaneously, the legs 112 on the gripper 24 engage the outer surface of the fixture mount or implant and firmly hold it in the housing. The implant is now ready to be safely stored or transported in the vial.

As yet another advantage, the housing 12 can be formed of a clear polymer or glass. A visual inspection through the housing reveals if the gripper is engaged with the implant. If the gripper is engaged with the implant, then the vial is in the closed position and the implant is not ready to be removed from the housing. On the other hand, if the gripper is not engaged with the implant, then it is ready to be removed from the housing.

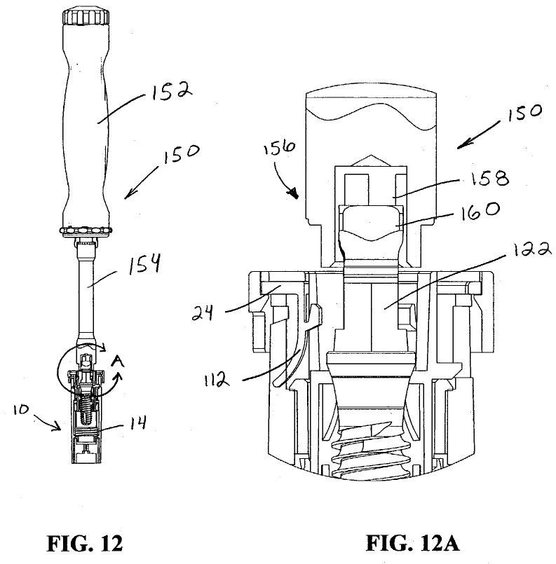

FIGS. 12 and 12A show a dental driving tool 150 connected to the end of fixture mount 122. The driving tool has a handle 152, an elongated drive shaft 154, and an engaging mechanism 156 at the distal end. The engaging mechanism includes a hexagonal recess 158 that is adapted to engage a hexagonal projection 160 at the end of the fixture mount 122.

In use, the engaging mechanism 156 engages the fixture mount 122 (or implant 120 if no fixture mount is used). As downward force is applied to the driving tool and fixture mount, the biasing member 14 compresses and the implant moves downwardly in the vial 10. Simultaneously, the legs 112 of the gripper 24 retract or move inwardly and away from the fixture mount and implant. The implant is thus disengaged or unlocked from the vial and can be readily pulled upwardly and out of the vial with the driving tool. In this regard, the engaging mechanism 156 has a retentive feature that is strong enough to hold the implant and carry it to the implantation site. Preferably, the driving tool holds the implant with a relatively slight retentive force. This removal force will not disrupt the position of the implant or otherwise deleteriously affect the commencement of osseointegration, even in soft bone of the posterior maxilla, when the driving tool disengages from the implant.

An important advantage of the present invention is that the vial has a retention mechanism that can be actuated to one of two positions. In one position, the implant is firmly held and maintained in the housing of the vial. In a second position, the implant is free to exit through an opening in the housing. The present invention, thus, comprises an active or moveable retention mechanism to hold and release the implant. This retention mechanism can have various configurations and still perform the function of holding and releasing the implant. FIGS. 1-12 illustrate a preferred embodiment for this mechanism, though other embodiments or variations are also within the scope of the invention. These variations are numerous, and one skilled in the art will contemplate such variations after reading this disclosure. By way of example, the biasing member and piston could be formed of a single unit or piece instead of two separate components. Further, the gripper and cradle could be formed as a single unit or piece instead of two separate components. As another example, the retention mechanism could be altered so the biasing member is compressed in the closed position and uncompressed in the open position. In general, the components of the retention mechanism could be altered, omitted, or combined; and other, new components could be added while such embodiments would not depart from the scope of the invention.

The vial and its components can be formed from various materials known to those skilled in the art. Preferably, these materials are biocompatible and include metals (such as titanium and titanium alloys) and various polymers acceptable for use in implant dentistry. The biasing member, for example, can be formed from steel while the other components of the vial are injection molded from polymer. Further, the dental driving tool, the implant, and the fixture mount can have various configurations known to those skilled in the art. By way of example, Centerpulse Dental Inc. of Carlsbad, Calif. manufactures driving tools, implants, and fixture mounts that can be employed with the present invention.

A method utilizing the present invention is discussed in detail. The vial of the present invention may be utilized with both edentulous sites and extraction sites. Further, such sites may be single or multiple restorations. For illustrative purposes, the accompanying description teaches application of the present invention to a single tooth extraction implantation site, but one skilled in the art will contemplate other uses as well.

The next steps involve placing the implant into the implantation site. Various implants may be placed into the osteotomy, but, preferably, the implant should be dimensioned to sufficiently fill the entire cervical region of the socket. One example of an implant appropriate for the method of the present invention is a Spline® implant, manufactured by Centerpulse Dental Inc. of Carlsbad, Calif.

The implant vial is removed from any external packaging, if such packaging is provided. Next, the coronal end of the implant is exposed while still being held and retained in the inner cavity of the vial. In this latter step, various devices may be used to cover and seal the implant in the vial. A cap, for example, may be unscrewed or otherwise removed from the top of the vial to expose the coronal end of the implant.

Next, a dental driving tool is obtained, and the driving end of this tool is engaged on the coronal end of the implant. One skilled in the art will appreciated that the implant may have a fixture mount transfer (FMT) connected to the coronal end. For purposes of this procedure, a FMT will not be used or described.

While the driving tool is engaged to the coronal end of the implant, a downward force is applied to the implant along its longitudinal axis. This force moves the implant toward the bottom of the vial. As the implant moves, the retention mechanism holding the implant disengages. Specifically, the implant is out of frictional engagement with the vial. All arms move away from or disengage with the external surface of the implant. At this point, the implant is not being firmly held in the vial; instead, the implant is free to move out through the open end of the internal cavity.

In the next step, the implant is moved upwardly and pulled out through the open end of the vial with the driving tool. The implant is carried to the implantation site, and the coronal end is driven into the osteotomy using the driving tool. The driving tool is then disengaged from the implant and removed from the implantation site.

Although illustrative embodiments have been shown and described, a wide range of modifications, changes, and substitutions is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

What is claimed is:

1. A vial for housing a dental implant, comprising:
a housing having a closed end and forming an internal cavity for housing the dental implant; and
a retention mechanism located in the housing and moveable to first and second positions, in the first position the retention mechanism engages the dental implant in the housing and prevents the implant from being removed from the housing, and in the second position the retention mechanism moves out of engagement with the implant so the implant can be removed from the housing.

2. The vial of claim 1 in which the retention mechanism further includes a gripper, wherein the gripper holds the implant in the first position and releases the implant in the second position.

3. The vial of claim 2 in which the gripper has flexible legs that engage the implant in the first position and disengage the implant in the second position.

4. The vial of claim 2 in which the retention mechanism further includes a biasing member that is moveable between the first and second positions.

5. The vial of claim 4 in which the biasing member is unbiased in the first position and compressed in the second position.

6. The vial of claim 5 in which the gripper includes flexible legs that disengage the implant as the biasing member is compressed.

7. The vial of claim 1 in which the retention mechanism further includes a gripper adapted to engage an outer surface of the dental implant in the first position and disengage from the outer surface as the retention mechanism is moved from the first position to the second position.

8. A vial for housing a dental implant, comprising:
a housing forming an internal cavity adapted to house the dental implant; and
a retention mechanism located in the housing, having a first position that grips an outer surface of the dental implant and holds it in the housing, and having a second position disengaged from the outer surface of the dental implant, wherein the retention mechanism is moveable from the first position to the second position.

9. The vial of claim 8 in which the retention mechanism further includes a biasing member that is unbiased in the first position and biased in the second position.

10. The vial of claim 9 in which the retention mechanism further includes a gripper that grips the outer surface of the implant in the first position and disengages from the outer surface as the retention mechanism is moved from the first to second positions.

11. The vial of claim 10 in which the retention mechanism further includes an indexer and contra-indexer that lock together in the second position.

12. The vial of claim 11 in which the indexer and contra-indexer have teeth that lock together and hold the implant in the second position.

13. The vial of claim 8 in which the retention mechanism further includes a cradle adapted to hold the implant and a biasing member adapted to bias the implant in the housing.

14. The vial of claim 13 in which the implant vertically moves in the housing between the first and second positions.

15. The vial of claim 14 in which the implant firmly held in the housing in the first position to prevent the implant from being removed from the housing.

16. A vial for holding a dental implant, the vial comprising:
a housing forming a cavity adapted to receive the dental implant; and
a retention mechanism located in the housing and including a biasing member located at a distal portion of the housing and a gripper adapted to hold the implant.

17. The vial of claim 16 in which the gripper has flexible legs that engage and hold the implant.

18. The vial of claim 17 in which the legs are moveable from an engaging position with the implant to a non-engaging position with the implant.

19. The vial of claim 16 in which the implant moves downwardly in the housing as the biasing member is compressed and as the gripper disengages from the implant.

20. The vial of claim 16 in which the gripper holds the implant while the biasing member is unbiased and releases the implant while biasing member moves to a biased position.
